(19) United States
(12) Patent Application Publication
(10) Pub. No.: US 2025/0032428 A1
(43) Pub. Date: Jan. 30, 2025

(54) COMPOSITIONS FOR MAINTAINING EYELID HYGIENE

(71) Applicant: OCuSOFT, Inc., Rosenberg, TX (US)

(72) Inventors: Nat Adkins, JR., Richmond, TX (US); Cynthia Barratt, Richmond, TX (US); Paramita Sarkar, Richmond, TX (US)

(21) Appl. No.: 18/917,883

(22) Filed: Oct. 16, 2024

Related U.S. Application Data
(63) Continuation-in-part of application No. 18/639,128, filed on Apr. 18, 2024.
Application No. 18/639,128 is a continuation-in-part of application No. 17/213,986, filed on Mar. 26, 2021, now abandoned.
Application No. 17/213,986 is a continuation-in-part of application No. 16/508,626, filed on Jul. 11, 2019, now Pat. No. 10,959,940.
(60) Provisional application No. 63/497,369, filed on Apr. 20, 2023.
(60) Provisional application No. 62/697,213, filed on Jul. 12, 2018.

(51) Int. Cl. A61K 31/133; A61K 9/00; A61K 31/047; A61K 31/155; A61K 31/164; A61K 47/10; A61K 47/18; A61K 47/26

(52) U.S. Cl. CPC A61K 31/133; A61K 9/0048; A61K 31/047; A61K 31/155; A61K 31/164; A61K 47/10; A61K 47/18; A61K 47/26

(57) ABSTRACT

Compositions and methods for cleansing the eyelid and treating an ocular condition with an ophthalmic composition consisting essentially of water, PEG-80 sorbitan laurate, methyl gluceth-20, PEG-120 methyl glucose dioleate, phytosphingosine HCl, decyl glucoside, 1,2-hexanediol, caprylyl glycol, disodium cocoamphodiacetate, panthenol, polyaminopropyl biguanide, sodium chloride, potassium chloride and calcium chloride.

### CROSS REFERENCE TO RELATED APPLICATIONS

This application claims priority to and is a continuation in part of U.S. patent application Ser. No. 18/639,128, filed on Apr. 18, 2024 which claims priority to U.S. Provisional Patent Application Ser. No. 63/497,369 filed on Apr. 20, 2023 and which is also a continuation-in-part of U.S. patent application Ser. No. 17/213,986 filed on Mar. 26, 2021 which is a continuation-in-part of U.S. Ser. No. 16/508,626 filed on Jul. 11, 2019 which claims the benefit of Provisional U.S. Patent Application No. 62/697,213, filed Jul. 12, 2018, the entire contents and disclosures of all of which, both express and implied, are incorporated herein by reference.

### BACKGROUND

Ocular health refers to eyes as well as structures associated with the eyes, eyelids for example. The eyelids are important in over-all ocular health because they protect the eyes from dangers such as approaching objects or from airborne contaminants, such as pollen, dust particles or other foreign bodies. The eyelids contain essential glands; the lacrimal glands and meibomian glands that produce layers of tear film that are critical for healthy eyes. When an individual blinks, a new tear film is created and tears are distributed across the cornea to lubricate the surface of the eye. This blinking action also “flushes” foreign materials from the eye.

The eyelids, however, are subject to certain problems, which while very common, are none-the-less bothersome, especially for contact lens wearers, and may lead to other more serious complications. One complication is blepharitis. Blepharitis is a common chronic inflammation of the eyelids characterized by a scaly crust on the lid margins. The condition may be caused by a bacterial infection, or it may be allergic in origin or associated with seborrhea of the face and scalp. Treatment usually involves cleansing the eyelids on a regular basis to remove excess oil, debris, and desquamated skin that may be problematic.

Often associated with or secondary to blepharitis is a bacterial infection of the surface of the skin at the edge of the lid know as an internal hordeolum. Other such infections include external hordeolum, commonly referred to styes, which are infections of the tiny oil secreting meibomian glands along the edge of the eyelid, surrounding the eyelashes. A stye begins as a red, tender bump and usually fully develops within three days. Such conditions are accompanied by pain, redness and tenderness of the eyelid (or lid) margins. Although styes are often recurring, regular cleansing of the eyelid margins can minimize such conditions. A second problem is a chalazion, which is an inflammation of the meibomian glands inside the eyelid. Chalazia typically grow slowly over 2-3 weeks and although they do not typically cause pain, they often require surgical intervention if left untreated.

With any of the above-described problems, as well as other medical complications, such as rosacea and seborrhea, proper eyelid hygiene with the use of an eyelid cleanser may minimize the severity of the outbreak or prevent the problem altogether if caught early. Eyelid cleansers are also used for cleaning eyelashes, eyelids or the periocular area and may be used as a pre-operative scrub to help reduce the presence of harmful bacteria which may cause infection, inflammation, or even endophthalmitis in patients.

### SUMMARY

The present application is directed to an ophthalmic composition consisting essentially of phytosphingosine or phytosphingosine HCl, PEG-120 methyl glucose dioleate, calcium chloride, potassium chloride, sodium chloride, decyl glucoside, PEG-80 sorbitan laurate, disodium cocoamphodiacetate, methyl gluceth-20, panthenol, 1,2-hexanediol, caprylyl glycol, polyaminopropyl biguanide, and purified water.

In a specific embodiment, the phytosphingosine or phytosphingosine HCl is present in a concentration ranging from 0.01 to 1% w/w and, more preferably, in a concentration ranging from 0.01 to 0.3% w/w.

According to embodiments of the ophthalmic composition, the composition consists essentially of 0.005 to 0.5% w/w calcium chloride, 0.005 to 0.5% w/w potassium chloride, and 0.05 to 1.2% w/w sodium chloride.

In yet another embodiment, the ophthalmic composition has a pH in the range of 5.5 to 7.5.

The ophthalmic composition consists essentially of a foam. In one embodiment, the ophthalmic composition may be dispensed as a pre-lathered foam.

According to a specific embodiment, the ophthalmic composition consists essentially of 0.2% phytosphingosine HCL, 3% PEG-120 methyl glucose dioleate, 0.03% calcium chloride, 0.03% potassium chloride, 0.7% sodium chloride, 0.5% decyl glucoside, 5.6% PEG-80 sorbitan laurate, 0.215% disodium cocoamphodiacetate, 4.3% methyl gluceth-20, 0.2% panthenol, 0.2% 1,2-hexanediol, 0.2% caprylyl glycol, 0.2% polyaminopropyl biguanide, and q.s. to 100% purified water.

In yet another embodiment, a kit for maintaining eyelid hygiene consists essentially of: the ophthalmic composition, wherein the ophthalmic composition consists essentially of phytosphingosine or phytosphingosine HCl, PEG-120 methyl glucose dioleate, calcium chloride, potassium chloride, sodium chloride, decyl glucoside, PEG-80 sorbitan laurate, disodium cocoamphodiacetate, methyl gluceth-20, panthenol, 1,2-hexanediol, caprylyl glycol, polyaminopropyl biguanide, and purified water; a pharmaceutically acceptable container for containing and dispensing the ophthalmic composition; and an applicator for applying the ophthalmic composition to an eyelid. The ophthalmic composition may have a volume of 25 ml to 100 ml. In a specific aspect, the ophthalmic composition may have a volume of 50 ml. In one aspect, the applicator comprises a plurality of fabric pads. In a specific aspect, the kit consists essentially of a 50 ml bottle of the composition and 100 dry lint-free fabric pads.

In another embodiment, the present application is directed to a method for maintaining eyelid hygiene. The method involves cleansing an eyelid with the ophthalmic composition and leaving the ophthalmic composition on the eyelid without rinsing, wherein the ophthalmic composition consists essentially of phytosphingosine or phytosphingosine HCl, PEG-120 methyl glucose dioleate, calcium chloride, potassium chloride, sodium chloride, decyl glucoside, PEG-80 sorbitan laurate, disodium cocoamphodiacetate, methyl gluceth-20, panthenol, 1,2-hexanediol, caprylyl glycol, polyaminopropyl biguanide, and purified water.

According to an embodiment, a method for preparing an ophthalmic composition suitable for maintaining eyelid hygiene comprises adding a first portion of purified water to a first mixing vessel, mixing 1,2-hexanediol, caprylyl glycol, and phytoshpingosine HCl in the first mixing vessel, adding a second portion of purified water to a second mixing vessel, mixing calcium chloride, potassium chloride, sodium chloride, panthenol, and PEG-120 methyl glucose dioleate into the second mixing vessel, transferring the contents of the first mixing vessel into the second mixing vessel, and mixing PEG-80 sorbitan laurate, disodium cocoamphodiacetate, methyl gluceth-20, and polyaminopropyl biguanide into the second mixing vessel.

### BRIEF DESCRIPTION OF THE DRAWINGS

FIG. 1 illustrates a kit according to an embodiment of the invention.

### DETAILED DESCRIPTION

While the specification concludes with the claims particularly pointing and distinctly claiming the invention, it is believed that the present invention will be better understood from the following description. All percentages and ratios used herein are by weight of the total composition. The present invention can comprise or consist essentially of the components of the present invention as well as other ingredients or elements described herein. As used herein, “comprising” means the elements recited, or their equivalent, plus any other element or elements which are not recited. The terms “having,” “including,” and “comprised of” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Generally, such additives may not be present at all or only in trace amounts. However, it may be possible to include up to about 10% by weight of materials that could materially alter the basic and novel characteristics of the invention as long as the utility of the composition (as opposed to the degree of utility) is maintained.

All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value.

As used herein, the term “effective amount” of a composition refers to an amount sufficient to elicit the desired biological response. In some embodiments, a therapeutically effective amount of a composition is an amount that is sufficient, when administered to a subject suffering from or susceptible to a disease, disorder, and/or condition, to treat, diagnose, prevent, and/or delay the onset of one or more symptoms of the disease, disorder, and/or condition. As will be appreciated by those of ordinary skill in this art, and effective amount of a substance may vary depending on such factors as the desired biological endpoint, the patient, etc. For example, the effective amount of a composition and/or formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition.

According to an embodiment, an ophthalmic composition is configured for a daily eyelid hygiene management regime. This composition is formulated as a non-irritating leave-on eyelid cleanser. Additionally, the ophthalmic composition includes both anti-bacterial and anti-inflammatory properties. It can also aid in wound-healing of the eyelids. The ophthalmic composition is suitable for topical application. Topical application is understood to comprise cosmetic and/or dermatological application on the eyelids. The formulation can be used to not only treat the source of most eyelid conditions, but also the resulting symptoms leading to improved patient compliance. The ophthalmic composition is formulated to effectively eradicate multiple different strains of bacteria commonly found on the eyelids. For example, the ophthalmic composition can eradicate at least seven different strains of bacteria including methicillin resistant Staphylococcus aureus (MRSA) and Staphylococcus epidermidis (Staph epi). The ophthalmic composition is formulated to therapeutically relieve irritation by removing oil, debris, pollen and other contaminants. It also has added anti-inflammatory properties for long term relief from symptoms.

In one embodiment, the ophthalmic composition consists essentially of phytosphingosine or phytosphingosine HCl, PEG-120 methyl glucose dioleate, calcium chloride, potassium chloride, sodium chloride, decyl glucoside, PEG-80 sorbitan laurate, disodium cocoamphodiacetate, methyl gluceth-20, panthenol, 1,2-hexanediol, caprylyl glycol, polyaminopropyl biguanide, and purified water.

For the purposes of this invention, polyaminopropyl biguanide is pseudonymous for polyhexamethylene biguanide, polyhexamethylene biguanide, and polyhexamethylene biguanide hydrochloride. Polyaminopropyl biguanide is an exemplary antimicrobial and preservative agent with significant activity against microbes. Further, the agent is ideal for use in the ocular area because it is compatible with a wide range of composition excipients, surfactant systems, and vehicles.

The ophthalmic composition includes phytosphingosine (PSG), or a salt thereof, as an anti-inflammatory and anti-irritant agent. The term “anti-irritant”, as used herein, is an agent that prevents or reduces soreness, roughness, or inflammation of a bodily part, such as, the eyelids. PSG is a natural chemical that is a part of the lipid family. PSG is a water-binding agent that mimics the natural lipid layer of the outer epidermis for increased moisturizing throughout the day. PSG has anti-redness and skin firming properties and can inhibit microorganisms. Beneficially, PSG has both anti-bacterial and wound-healing properties and it acts as an anti-inflammatory at concentrations as low as 0.1%. In one aspect, the ophthalmic composition consists essentially of PSG between 0.01-1% by weight and preferably, 0.01-0.3% by weight.

The 1,2-glycol compounds can include 1,2-hexanediol and caprylyl glycol, also known as 1,2-octanediol. The diols can be included in the composition to enhance antimicrobial activity. Importantly, the combination of the diols and polyaminopropyl biguanide creates a synergistic antimicrobial effect.

The surfactants in the ophthalmic composition include a combination of nonionic surfactants and at least one of amphoteric or anionic surfactants. Suitable amphoteric surfactants include, but are not limited to alkyldimethyl betaines, alkylamido betaines, sulfobetaines, and imidazoline amphoterics. Suitable anionic surfactants include, but are not limited to fatty alcohol sulfates, alpha olein sulfonates, sulfosuccinates, sarcosinates, phosphate esters, and carboxylates. Suitable nonionic surfactants include, but are not limited to alkanolamides, ethoxylate amides, esters, alkylated alcohols, alkylpolyglucosides, amine oxides, sorbitan esters, and ethoxylates.

Polyaminopropyl biguanide is most effective as an antimicrobial agent in pH ranges between 5.5 and 7.5. Therefore, it is desirable to control the pH of the ophthalmic composition within this range by use of a blend of surfactants. It is also desirable that the ophthalmic composition has a foaming ability to facilitate physical cleansing of the eyelid. Consequently, surfactants must be chosen which will both control the pH of the ophthalmic composition within polyaminopropyl biguanide's effective range and provide the foaming ability necessary to physically clean the eyelid.

Advantageously, the ophthalmic composition does not include traditional pH adjusters which can be irritating to the eye. In general, surfactants are less irritating to the eye than traditional pH adjusters. Examples of traditional pH adjusters, include basic pH adjusters, such as ammonia, mono-, di- and tri-alkyl amines, mono-, di- and tri-alkanolamines, alkali metal and alkaline earth metal hydroxides (e.g., ammonia, sodium hydroxide, potassium hydroxide, lithium hydroxide, monoethanolamine, triethylamine, isopropylamine, diethanolamine and triethanolamine), and acidic pH adjusters, such as mineral acids and polycarboxylic acids (e.g., hydrochloric acid, nitric acid, phosphoric acid, sulfuric acid, citric acid, glycolic acid, and lactic acid).

In a specific embodiment, the ophthalmic composition includes disodium cocoamphodiacetate, PEG-80 sorbitan laurate, and decyl glucoside. Disodium cocoamphodiacetate is an amphoteric surfactant. PEG-80 sorbitan laurate and decyl glucoside are both nonionic surfactants. Surfactants can reduce irritation and can increase the cleansing ability of the ophthalmic composition and provide it with a foaming capability. Foams are considered to have optimal cleansing and preventive capability.

The combination of surfactants is added to a modified Ringer's solution. For the purposes of this invention, a modified Ringer's solution is an isotonic aqueous solution of electrolytes which is physiologically compatible with human tissue. In one embodiment, the modified Ringer's solution comprises sodium chloride, potassium chloride, calcium chloride, and water. The modified Ringer's solution is included in the surfactant solution to ensure that the composition will not remove water from the eyelids by osmosis. Preferably, the water used is purified water. The modified Ringer's solution may also comprise 0.05 to 1.2 wt. % sodium chloride, 0.005 to 0.5 wt. % potassium chloride, 0.005 to 0.5 wt. % calcium chloride, and water. In still another embodiment, the modified Ringer's solution comprises about 0.7 wt. % sodium chloride, about 0.03 wt. % potassium chloride, about 0.03 wt. % calcium chloride, and purified water.

Moisturizers are chemicals that prevent transepidermal water loss and restore moisture to the skin. Moisturizers may prevent water loss by forming a film over the skin to prevent water from evaporating from the skin. Alternatively, moisturizers comprise hydroscopic molecules that draw water from the air into the skin. In one embodiment, the ophthalmic composition includes a suitable moisturizer such as, but not limited to, methyl gluceth-20, sorbital, glycerine, propylene glycol, carboxylates, amino acids, glucoside derivatives, urea, lactates, and derivatives of pantothenic acid. Examples of derivatives of pantothenic acid include panthenol, D-panthenol, and D,L-panthenol.

In an embodiment, the ophthalmic composition also includes a foam stabilizer. A foam stabilizer is a surfactant that has both hydrophilic and hydrophobic properties. The hydrophobic portion of the foam stabilizer interacts with the air at the surface of the composition, while the hydrophilic part interacts with the aqueous portion. The foam stabilizer can be a polyethylene glycol diester of methyl glucose and a fatty acid. Suitable fatty acids include oleic acid, steric acid, lauric acid, caprylic acid, and capric acid. Suitably, the foam stabilizer is PEG-120 methyl glucose dioleate.

A specific embodiment of the ophthalmic composition consists essentially of purified water, 0.2% phytosphingosine HCl, 5.6% PEG-80 sorbitan laurate, 4.3% methyl gluceth-20, 3% PEG-120 methyl glucose dioleate, 0.5% decyl glucoside, 0.2% 1,2-hexanediol, 0.2% caprylyl glycol, 0.215% disodium cocoamphodiacetate, 0.2% panthenol, 0.2% polyaminopropyl biguanide, 0.7% sodium chloride, 0.03% potassium chloride and 0.03% calcium chloride. The ingredients selected in this embodiment specifically ensure that the formulation possesses anti-bacterial, anti-inflammatory and wound-healing capabilities.

The ophthalmic composition is effective as a scrub, as it has an antimicrobial effect, but is still practically non-irritating to the eye. The ophthalmic formulation has these beneficial characteristics because of the combination of polyaminopropyl biguanide, 1, 2-glycol compounds and the sphingoid lipid. Advantageously, the formulation does not contain astringents such as, zinc or zinc salts. Examples of zinc salts include zinc acetate, zinc lactate, zinc gluconate, zinc citrate, zinc butyrate, and zinc sterate.

According to an embodiment, a method of cleansing the eyelid comprises providing the ophthalmic composition disclosed herein. The method further involves cleansing the eyelids with an effective amount of the formulation. The ophthalmic composition can be allowed to remain on the eyelid after cleansing without rinsing. This is because the ophthalmic composition is formulated to be mild enough to be left on the eyelid. The ability of the ophthalmic composition to be left on the eyelid rather than rinsed off increases the formulation's anti-microbial effect. In general, the longer an anti-microbial composition is allowed to contact the pathogens, the more pathogens it will kill.

In another aspect of the method of cleansing the eyelid, the ophthalmic composition is applied to the eyelid from an applicator. The applicator may be a dry and substantially lint-free fabric pad, such as, a rayon pad or a rayon and polypropylene fabric blend. The fabric pad can comprise a textured surface. The ophthalmic composition may be rubbed on the eyelid with the fabric pad to induce foaming, which assists in the cleansing ability of the formulation. In one embodiment, the ophthalmic composition may be combined with the fabric pad to form an apparatus for cleansing the eyelids. In yet another embodiment, an eyelid cleansing apparatus comprises a fabric pad that is pre-moistened with the ophthalmic composition.

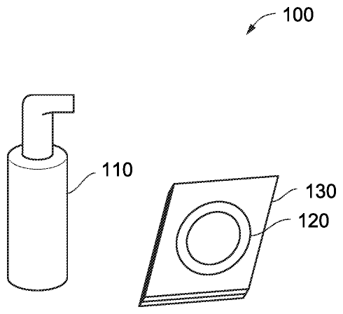

In another embodiment, as shown in FIG. 1, an eyelid cleansing kit 100 comprises a pharmaceutically acceptable container/dispensing device 110. A predetermined amount of the ophthalmic composition is contained inside the container 110. The kit further comprises an applicator 120.

The container 110 may be a pump dispenser known in the art. The container 110 can be configured to deliver the ophthalmic composition as pre-lathered foam without requiring any outside physical or mechanical action to prevent loss of the composition. For convenience and economy, the container 110 may be configured to contain about 25 ml to 100 ml of the ophthalmic composition. Preferably, the container 110 may be configured to contain about 50 ml of the ophthalmic composition.

The applicator 120 is configured for receiving the ophthalmic composition. In one aspect, the applicator 120 includes one or more absorbent pads. The pads 120 comprise a lint-free fabric, such as, rayon or another suitable material that can receive the ophthalmic composition. The pads 120 may be single use disposable pads. The eyelid cleansing kit 100 can include between 1-100 pads 120. In one embodiment, one or more of the pads 120 may be contained within a sealable container 130. In one aspect, the sealable container 130 may comprise a box or a package. The package may be made of any suitable material including plastic or metal foil material. The pump dispenser 110 may also be enclosed in suitable housing (not shown). In another aspect, the applicator 120 includes one or more swabs or swab sticks (not shown). In another aspect of the invention, paper towels, cotton balls or even the fingertips can be employed to apply the ophthalmic composition to the eyelid.

In another embodiment, the kit consists essentially of a plurality of fabric pads pre-moistened with the ophthalmic composition. The pre-moistened fabric pads may be individually packaged for use.

Blepharitis is a common eye disorder characterized by an inflammation of the eyelids that causes red, irritated, itchy eyelids and the formation of dandruff-like scales on eyelashes. Blepharitis may be caused by bacteria or skin conditions, such as, dandruff of the scalp or acne rosacea. Anterior blepharitis occurs at the outside front edge of the eyelid where the eyelashes are attached. The root cause of anterior blepharitis is the overproduction of oils. The ophthalmic formulation is effective for daily use in all blepharitis conditions. However, in the most severe conditions, it may be used in combination with a second ophthalmic formulation containing hypochlorous acid to optimally treat severe cases of anterior blepharitis.

### Method of Preparation

The method described hereafter is suitable for producing an ophthalmic composition configured for maintaining eyelid hygiene. To a first mixing vessel, add a first portion of purified water followed by 1,2-hexanediol and caprylyl glycol. Under continuous mixing conditions, then add phytosphingosine HCl slowly and away from the walls of the first mixing vessel. Mixing should proceed until the solution is clear of undissolved phytosphingosine HCl.

To a second mixing vessel, add a second portion of purified water. Sequentially add to the second mixing vessel calcium chloride, potassium chloride, and sodium chloride. Each salt should be added slowly and away from the walls of the second mixing vessel and be fully dissolved, through continuous mixing, prior to the addition of the subsequent ingredients. Similarly, add panthenol to the second mixing vessel. PEG-120 methyl glucose dioleate is then added to the second mixing vessel with mixing proceeding until the foam stabilizer is completely dispersed and entrapped air is substantially reduced. Manual scraping of the walls of the second mixing vessel may be required for complete dispersion.

Transfer and mix the contents from the first mixing vessel into the second mixing vessel. PEG-80 sorbitan laurate, disodium cocoamphodiacetate, methyl gluceth-20, and polyaminopropyl biguanide are then added separately and sequentially to the second mixing vessel while continuously mixing. Mixing is complete when the solution is completely dispersed and uniform.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

1. An ophthalmic composition consisting essentially of:
Phytosphingosine HCl
PEG-120 methyl glucose dioleate
Calcium chloride
Potassium chloride
Sodium chloride
Decyl glucoside
PEG-80 sorbitan laurate
Disodium cocoamphodiacetate
Methyl gluceth-20
Panthenol
1,2-hexanediol
Caprylyl glycol
Polyaminopropyl biguanide
Purified water

2. The ophthalmic composition according to claim 1, wherein the phytosphingsine HCl is present in a concentration ranging from 0.01 to 1% w/w.

3. The ophthalmic composition according to claim 1, wherein the phytosphingsine HCl is present in a concentration ranging from 0.01 to 0.3% w/w.

4. The ophthalmic composition according to claim 1, wherein calcium chloride is present in a concentration ranging from 0.005 to 0.5% w/w.

5. The ophthalmic composition according to claim 1, wherein potassium chloride is present in a concentration ranging from 0.005 to 0.5% w/w.

6. The ophthalmic composition according to claim 1, wherein sodium chloride is present in a concentration ranging from 0.05 to 1.2% w/w.

7. The ophthalmic composition according to claim 1, wherein the pH is in the range of 5.5 to 7.5.

8. The ophthalmic composition according to claim 1, wherein composition consists essentially of a foam.

9. An ophthalmic composition consisting essentially of:
0.2% Phytosphingosine HCl
3% PEG-120 methyl glucose dioleate
0.03% Calcium chloride
0.03% Potassium chloride
0.7% Sodium chloride
0.5% Decyl glucoside
5.6% PEG-80 sorbitan laurate
0.215% Disodium cocoamphodiacetate
4.3% Methyl gluceth-20
0.2% Panthenol
0.2% 1,2-hexanediol
0.2% Caprylyl glycol
0.2% Polyaminopropyl biguanide
q.s. to 100% Purified water.

10. A kit for maintaining eyelid hygiene consisting essentially of:
the ophthalmic composition according to claim 1;
a pharmaceutically acceptable container for containing ophthalmic composition, and
an applicator for applying the ophthalmic composition to an eyelid.

11. The kit according to claim 10, wherein the ophthalmic composition has a volume of 25 ml to 100 ml.

12. The kit according to claim 10, wherein the applicator comprises a plurality of fabric pads.

13. A method for maintaining eyelid hygiene comprising:
providing the ophthalmic composition according to claim 1;
cleansing an eyelid with the ophthalmic composition; and
leaving the ophthalmic composition on the eyelid without rinsing.

14. A method for preparing an ophthalmic composition comprising:
adding a first portion of purified water to a first mixing vessel;
mixing 1,2-hexanediol, caprylyl glycol, and phytosphingosine HCl into the first mixing vessel;
adding a second portion of purified water to a second mixing vessel;
mixing calcium chloride, potassium chloride, sodium chloride, panthenol, and PEG-120 methyl glucose dioleate into the second mixing vessel;
transferring the contents of the first mixing vessel into the second mixing vessel; and
mixing PEG-80 sorbitan laurate, disodium cocoamphodiacetate, methyl gluceth-20, and polyaminopropyl biguanide into the second mixing vessel.
